(19) United States
(12) Patent Application Publication
(10) Pub. No.: US 2025/0201391 A1
(43) Pub. Date: Jun. 19, 2025

(54) PERSISTENT HEALTH MONITORING OF A PATIENT BY A MEDICAL SYSTEM INVOKING AN APPLICATION RESTART

(71) Applicant: Medtronic, Inc., Minneapolis, MN (US)

(72) Inventors: Eric D. Dorphy, Maple Grove, MN (US); Matthew R. Yoder, Franklin, WI (US)

(21) Appl. No.: 18/849,563

(22) PCT Filed: Mar. 15, 2023
(86) PCT No.: PCT/IB2023/052514

§ 371 Date: Sep. 23, 2024

Related U.S. Application Data
(60) Provisional application No. 63/269,820, filed on Mar. 23, 2022.

(51) Int. Cl. G16H 40/20; A61B 5/11; G01S 19/14; G16H 10/60

(52) U.S. Cl. CPC G16H 40/20; A61B 5/1112; A61B 5/1113; G16H 10/60; G01S 19/14

(57) ABSTRACT

This disclosure is directed to medical systems and techniques for enhanced health monitoring using location information. In one example, a method performed by processing circuitry of a computing device is described. The method comprises: determining that a monitoring application, previously executed by the processing circuitry, is inoperative; receiving, from an input device of the computing device, input data indicative of a current location of a patient; determining whether the current location of the patient corresponds to an authenticated area of the patient; and in response to the determination that the current location corresponds to the authenticated area and to the determination that the monitoring application is inoperative, restarting the monitoring application.

### FIELD

The disclosure relates generally to medical systems and, more particularly, medical systems configured to monitor patient activity for changes in patient health and/or to deliver therapy to a patient.

### BACKGROUND

Some types of medical systems may monitor various types of data of a patient or a group of patients for one or several purposes. Amongst the numerous examples, some medical systems may record measurements of a patient and their heart as indicia of cardiac health for that patient, which may be memorialized in raw data and/or processed data formats; as one example, electric signals representing cardiac activity over a period of time may be memorialized as a cardiac electrogram (EGM) and then, processed into other indicia of the cardiac health of the patient. In some examples, the medical system may monitor the EGM to detect one or more types of arrhythmia, such as bradycardia, tachycardia, fibrillation, or asystole (e.g., caused by sinus pause or AV block). In some examples, the medical system may include one or more of an implantable medical device or a wearable device to collect various measurements used to detect changes in patient cardiac health. In some examples, the medical system may deliver a therapy, and may record information regarding the delivery of therapy.

### SUMMARY

Medical systems and techniques as described herein facilitate administration of medical care to patients while continuing to monitor patient activity and detect changes in patient health (if any). The present disclosure describes “medical care” as referring to any product and/or process capable of making a patient more healthful and, typically, includes medical procedures, clinical evaluations, methods for providing therapy, methods involving diagnosis and/or treatment, healthcare-related processes, and/or the like. Administration of “medical care” for any given patient generally involves providing a health monitoring service with access to the patient via their personal device(s) and then, successfully performing the medical process and/or delivering the medical product.

Current methodologies for the administration of the patient's medical care have inefficiencies and/or latencies, possibly jeopardizing the patient's health. If the patient's personal device is shut down and/or a particular application intended to run on that personal device is determined to be inoperative, a medical system for the patient is limited in operation and, most likely, cannot transition to a next step in the patient's medical care.

There are a number of ways for the particular application to transition from operative to inoperative in status. The particular application may not be actively running on a computing device, or an operating system may run the particular application in a background of instead of a foreground (i.e., as a background application). The particular application may have been previously executed and running in a computing device (at some point in a recent past) but later became an inoperative application, for some reason(s) of which some examples include receiving an error, being inactive, and/or causing a fault. The operating system may deprioritize and, possibly, shut-down the particular application, for example, due to application inactivity in excess of a threshold amount of time. The operating system may automatically shut-down the deprioritized particular application to satisfy resource requirements of other applications. There may be a failure in one or more components of or in one or more resources consumed by the particular application, which causes the particular application to be inoperative. Upon becoming inoperative, the computing device may lose a network connection that the particular application had with a computing service, such as a health monitoring service, and the operating system may destruct the particular application, such as by deleting any application data from memory resources and deallocating processing resources. The particular application may be malfunctioning in some manner where no error code is displayed but the malfunction is clearly evident based on application activity/behavior.

In each of the above examples, the particular application becoming inoperative causes substantial delays and inefficiencies in the administration of medical care to the patient, and restarting the particular application mitigates those delays and inefficiencies. Otherwise, uploads of patient data from the medical device may be lost and/or communications between the health monitoring service and the patient device may cease. Regardless of how the particular application became inoperative, the medical systems and techniques described herein may initiate a mechanism for triggering execution of the particular application in order to advance/facilitate the administration of medical care to the patient.

In one example, a method performed by processing circuitry of a computing device is described. The method comprises: determining that a monitoring application, previously executed by the processing circuitry, is inoperative; receiving, from an input device of the computing device, input data indicative of a current location of a patient; determining whether the current location of the patient corresponds to an authenticated area of the patient; and in response to the determination that the current location corresponds to the authenticated area and to the determination that the monitoring application is inoperative, restarting the monitoring application.

In one example, a computing device is described comprising: an input device; a memory; and processing circuitry configured to execute logic for a monitoring application stored in the memory. The computing device further comprises processing circuitry further configured to: determine that a monitoring application, previously executed by the processing circuitry, is inoperative; receive, from an input device of the computing device, input data indicative of a current location of a patient; determine whether the current location of the patient corresponds to an authenticated area of the patient; and in response to the determination that the current location corresponds to the authenticated area and to the determination that the monitoring application is inoperative, restart the monitoring application.

In one example, a medical system is described. The medical system comprises processing circuitry executing logic stored in memory configured to: determine that a monitoring application, previously executed by the processing circuitry, is inoperative; receive, from an input device of the computing device, input data indicative of a current location of a patient; determine whether the current location of the patient corresponds to an authenticated area of the patient; and in response to the determination that the current location corresponds to the authenticated area and to the determination that the monitoring application is inoperative, restart the monitoring application.

In one example, a non-transitory computer-readable storage medium is described comprising program instructions that, when executed by processing circuitry of a medical device, cause the processing circuitry to: determine that a monitoring application, previously executed by the processing circuitry, is inoperative; receive, from an input device of the computing device, input data indicative of a current location of a patient; determine whether the current location of the patient corresponds to an authenticated area of the patient; and in response to the determination that the current location corresponds to the authenticated area and to the determination that the monitoring application is inoperative, restart the monitoring application.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with one or more examples of the present disclosure.

FIG. 2 is a block diagram illustrating an example configuration of a medical device, in accordance with one or more examples of the present disclosure.

FIG. 3 is a block diagram illustrating an example configuration of the external device of FIG. 1, in accordance with one or more examples of the present disclosure.

FIG. 4 is a block diagram illustrating an example architecture of the health monitoring service of FIG. 1 that is operative on the computing system of FIG. 1, which may be coupled to the medical device and external device of FIGS. 1-4, in accordance with one or more examples of the present disclosure.

FIG. 5 is a flow diagram illustrating an example operation for administering medical care in a geofenced geographic area, in accordance with one or more examples of the present disclosure.

Like reference characters denote like elements throughout the description and figures.

### DETAILED DESCRIPTION

In general, medical systems according to this disclosure implement techniques for authenticating a restart of an application after becoming inoperative at a computing device/system of a patient. The present disclosure describes various mechanisms for initiating functionality at the computing device/system, which the techniques described herein advantageously utilize to accomplish the authenticated application restart. These techniques allow the medical systems described herein to resume operation at the computing device without a significant lapse in administration of medical care for the patient. This is because any delay in such administration risks harm to the patient's health.

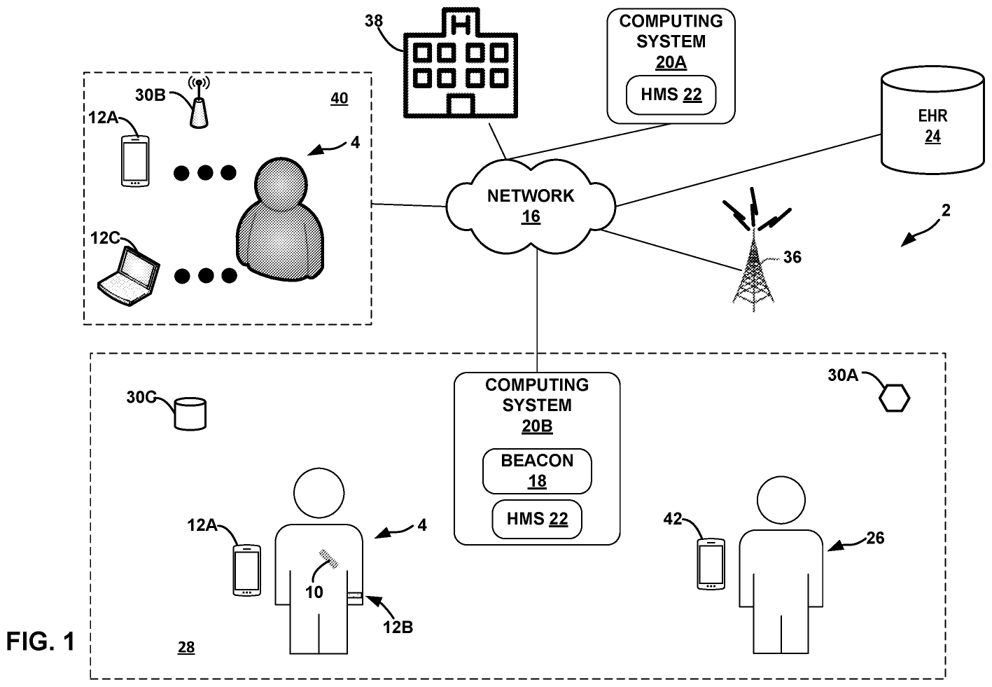

FIG. 1 is a block diagram illustrating an example system 2 configured detect health events of a patient 4, and to respond to such detection, in accordance with one or more techniques of this disclosure. As used herein, the terms “detect,” “detection,” and the like may refer to detection of a health event presently (at the time the data is collected) being experienced by patient 4, as well as detection based on the data that the condition of patient 4 is such that they have a suprathreshold likelihood of experiencing the health event within a particular timeframe. Examples of health events are a cardiac arrest, a ventricular fibrillation, a ventricular tachycardia, a stroke, a seizure, or a fall. The example techniques may be used with one or more patient sensing devices, e.g., implantable medical device (IMD) 10, which may be in wireless communication with one or more patient computing devices, e.g., patient computing devices 12A and/or 12B (herein “patient computing devices 12”). Although not illustrated in FIG. 1, IMD 10 may include electrodes and/or other sensors to sense physiological signals of patient 4, and may collect and store sensed physiological data based on the signals and detect episodes based on the data.

In some examples, IMD 10 may be implanted outside of a thoracic cavity of patient 4 (e.g., subcutaneously in the pectoral location illustrated in FIG. 1). In some other examples, IMD 10 may be implanted at other subcutaneous locations on patient 108 such as at an abdominal location. IMD 10 may be implanted subcutaneously or submuscularly on the left midaxillary of patient 4, such that IMD 10 may be positioned on the left side of patient 4 above the ribcage.

IMD 10 may be positioned near the sternum near or just below the level of the heart of patient 4, e.g., at least partially within the cardiac silhouette. In some examples, IMD 10 takes the form of the LINQ™ insertable cardiac monitor (ICM). Although described primarily in the context of examples in which IMD 10 takes the form of an ICM, the techniques of this disclosure may be implemented in systems including any one or more implantable or external medical devices, including monitors, sensors, pacemakers, defibrillators, wearable external defibrillators, neurostimulators, or drug pumps. Furthermore, although described primarily in the context of examples including a single implanted patient sensing device, in some examples a system includes one or more patient sensing devices, which may be implanted within patient 4 or external to (e.g., worn by) patient 4.

Given that, in general, computing device 12 represents any suitable embodiment of hardware/software that can be configured to execute and run a software application, patient 4 may select their personal device from a variety of computing device types. In the example illustrated by FIG. 1, computing device 12A may take the form of a smartphone, computing device 12B may take the form of a smartwatch or other smart apparel, and computing device 12C may take the form of any personal computer configured for wireless communication with IMD 10, such as a desktop, laptop, a notebook computer, tablet computer, workstation, one or more servers, cellular phone, or personal digital assistant. One or more of computing devices 12 may be configured to communicate with one or more computing systems, e.g., computing systems 20A and 20B (collectively, “computing systems 20”) via network 16.

Computing device(s) 12 may store in memory and/or transmit, via a network interface, various types of information as described herein. Examples of the types of data managed by computing device(s) 12 include sensed data, e.g., values of physiological parameters measured by IMD 10 and, in some cases one or more of computing devices 12, data regarding episodes of arrhythmia or other health events (initially) detected by IMD 10 and computing device(s) 12, and other physiological signals or data recorded by IMD 10 and/or computing device(s) 12. HMS 22 may also retrieve data regarding patient 4 from one or more sources of electronic health records (EHR) 24 via network. EHR 24 may include data regarding historical (e.g., baseline) physiological parameter values, previous health events and treatments, disease states, comorbidities, demographics, height, weight, and body mass index (BMI), as examples, of patients including patient 4. HMS 22 may use data from EHR 24 to configure algorithms implemented by IMD 10 and/or computing devices 12 to detect health events for patient 4. In some examples, HMS 22 provides data from EHR 24 to computing device(s) 12 and/or IMD 10 for storage therein and use as part of their algorithms for detecting health events.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

Computing system 20A may comprise, or may be implemented by, the Medtronic CareLink™ Network, in some examples. In the example illustrated by FIG. 1, computing system 20A implements a health monitoring service (HMS) 22, although in other examples, either or both of computing systems 20 may implement HMS 22. As will be described in greater detail below, HMS 22 may communicate a message instructing computing device(s) 12 to automatically restart a monitoring application that is determined to be inoperative, such as when the monitoring application is closed, not running or running in a background (e.g., dormant), malfunctioning (e.g., unpredictable behavior including false alarms), or otherwise is unable/unequipped to detect the false positive. The monitoring application may be a third-party application or a client for HMS 22 to run in a patient device.

Computing system 20B may include beacon device 18 (or simply beacon 18) as an example embodiment of a programmable beacon that a computing service may utilize to process location data indicative of a current location of beacon 18 and/or a current patient location. The computing service, which may be different from HMS 22, may be a location service configured to communicate the location data to beacon 18, prompting computing system 20B to communicate various messages to computing device(s) 12. These communications may be processed by a second application (i.e., a beacon application), and based on an operating status of the monitoring application, the second application may invoke a wake up function, which is a function call requesting an operating system execute program code of the monitoring application. In some examples, the programmable beacon may receive an operating status of the monitoring application and determine that the application has become inoperable. It should be noted that geofencing is not necessary in a number of examples. The patient device may derive a current location in a number of ways not involving geofencing.

Computing device(s) 12 may be configured for wireless communication with various devices including IMD 10, another computing device 12, computing systems 20, IoT devices 30, and/or the like. Computing devices 12 may communicate with the other devices via near-field communication (NFC) technologies (e.g., inductive coupling, NFC or other communication technologies operable at ranges less than 10-20 cm) and far-field communication technologies (e.g., radiofrequency (RF) telemetry according to the 802.11or Bluetooth® specification sets, or other communication technologies operable at ranges greater than near-field communication technologies). Computing devices 12 may communicate with a beacon device according to BLUETOOTH or BLUETOOTH Low Energy (BLE) protocols, as examples. In some examples, only one of computing devices 12, e.g., computing device 12A, is configured for communication with IMD 10, e.g., due to operation of hardware (e.g., as part of a BLE receiver/transmitter module) and/or due to execution of software (e.g., as part of the monitoring application described herein) enabling communication and interaction with IMD 10.

Various devices illustrated in the example of FIG. 1 may be configured as a beacon device similar to beacon 18 of computing system 20B. These devices, including local computing devices (such as those for computing system 20B or computing device 12C), external (non-patient) computing devices (such as those for computing system 20A), Internet of Things (IoT) devices 30, among other devices illustrated in FIG. 1 for an area, such as area 28 or area 40, may be configured to communicate with computing device(s) 12, for example, via wireless/wired network connectivity (e.g., a local area network or the Internet). These devices may operate independently from HMS 22 and considered “stand-alone” beacons for automatically restarting the monitoring application described herein.

Area 28 and area 40 may include one or more IoT devices, such as IoT devices 30A-30C (collectively “IoT devices 30”) illustrated in the example of FIG. 1. IoT devices 30 may include, as examples, so called “smart” speakers, cameras, lights, locks, thermostats, appliances, actuators, controllers, or any other smart home (or building) devices. In the example of FIG. 1, IoT device 30C is a smart speaker and/or controller, which may include a display. As described herein, any one or more of IoT devices 30 may operate as programmable beacon(s) configured to facilitate restarting of the monitoring application. IoT device 30 may communicate a message invoking a wake-up function for the monitoring application on computing device(s) 12A-12C after determining that the monitoring application is inoperative and authenticating the restart using location data. The wake-up function may be exposed by a beacon application corresponding to the beacon device of IoT device 30.

Area 40 includes a residence of patient 4 and area 28 includes a clinic used by patient 4. Each geographic area has necessary hardware devices/software programs to implement a local network by which computing device(s) 12, IoT device(s) 30, and other devices communicate with each other. Each geographic area also has necessary hardware devices/software programs for achieving network connectivity to remote networks. For example, area 28 may be configured with wireless technology, such as 802.11 wireless networks, 802.15 ZigBee networks, or the like. Area 28 may include one or more wireless access points that provide support for wireless communications throughout area 28. Additionally or alternatively, e.g., when local network is unavailable, computing devices 12, IoT devices 30, and other devices within area 28 may be configured to communicate with network 16, e.g., with HMS 22, via a cellular base station 36 and a cellular network.

HMS 22 is an example of a computing service that provides various computing resources (e.g., capacities and capabilities) over network 16, including remote monitoring of patient 4 and health event detection based on patient data. HMS may be considered a cloud computing service that provides additional storage, processing, and network resources to computing device(s) 12 and any other external device to IMD 10. HMS 22 may program hardware/software components of computing device(s) 12 to have patient data transmitted to HMS 22 via a connection that is either wireless or wired so that the data can be reviewed remotely by physicians, device clinic personnel, and device manufacturer personnel. Because access to the physical programmers and trained personnel is limited or non-existent in a retirement community, delays in device/patient assessment are common, leading to inefficiencies in patient care. To overcome the increased costs, the medical systems and techniques of the present disclosure enable persistent and continuous monitoring, which ensures access to and control of peri-procedural device interrogation/patient assessment over network 16. Some medical systems and techniques leverage various technologies to program beacon devices to automatically restart the monitoring application on a patient device based on current location (e.g., patient location and/or patient device location) and depending on requirements of HMS 22.

HMS 22 may be configured to receive patient data from IMD 10 and/or patient device(s) 12 by triggering execution of the monitoring application (e.g., if determined to be inoperative). Between HMS 22 and computing device(s) 12 of patient 4 in geographic area(s) 28 and/or 40, computing system(s) 20 facilitate remote monitoring for patient 4 on behalf of various entities. In area 40, computing system 20A may configure a computing service to operate on a local area network for computing device 12A, computing device 12C, and IoT device 30B; in so doing, computing system 20A may instrument IoT device 30A to output location data for the computing service and/or configure computing device 12C to communicate beacon messages to computing device 12A. In area 28, computing system 20B may configure a computing service on a local area network, for example, as a local service, or, alternatively, as part of a remote service running from computing system 20A.

HMS 22 may program a hardware/software component in computing device(s) 12, computing system(s) 20, IoT device(s) 30, and any other device in a given geographic area (e.g., area 40 of FIG. 1) to automatically restart the monitoring application whenever the application becomes inoperative and patient 4 is located within an authenticated area. For instance, computing device 12A may include a receiver/transmitter that can communicate with a programmable beacon device, such as beacon 18 of computing system 20B, and receive input data indicative of a current location. In response, computing device 12A may automatically restart the monitoring application and by way of that transmitter, communicate a message indicating a successful restart (e.g., an acknowledgment).

Patient 4 residing in area 40 desires receive medical care in the form of remote monitoring via HMS 22 and the monitoring application described herein. Remote monitoring, generally, refers to evaluations of patient data for different tasks, such as detecting various health events in patient 4. The monitoring application running in computing device 12A may facilitate the remote monitoring of patient 4 in a variety of ways. Medical systems and techniques described herein allow HMS 22 to enhance the care given patient 4 by adding beneficial features to such remote monitoring.

To illustrate one example feature to enhance the care given patient 4, consider patient 4 moving around different geographic areas, including their home/residence (e.g., a retirement community of area 40) and various medical facilities (e.g., hospital 38 or a clinic in area 28), where, depending on location data, HMS 22 may automatically restart a monitoring application that has become inoperative and/or non-responsive. The present disclosure construes certain geographic areas as being sufficiently tied to patient 4 to authenticate such a restart. For a number of reasons, patient 4's home in area 40 authenticates another party to exert control over the monitoring application. The present disclosure describes medical systems and techniques that make advantageous use of beaconing technologies and geofencing to accomplish the automatic restarts.

As described herein, HMS 22 may construct a geofence within one or more geographic areas to prompt an automatic restart for an inoperative (example/instance of the) monitoring application in each patient device. The geofence triggers execution of the monitoring application, which is configured to support the remote monitoring by HMS 22, for example, when patient 4 enters the one or more geofenced areas with the inoperative monitoring application or when the monitoring application stops working while patient 4 and their patient device are physically present in the one or more geofenced areas. In addition to or instead of geofencing, HMS 22 may avail other controls and methods for monitoring an environment within a geographic area, such as area 28, including an authenticated area, such as area 40.

The present disclosure generally describes an authenticated area as a geographic area that has been verified as a known patient location. Outside of the authenticated area(s), there is a substantially lower likelihood for patient presence, despite possibly being an actual patient location. Area 28 and area 40 represent an unauthenticated area and authenticated area, respectively. Therefore, a beacon message directed to computing device 12A in area 40, in contrast, succeeds in invoking a function call for the wake-up process (i.e., a wake-up function call) and triggering execution of the monitoring application. A beacon message directed to computing device 12A in area 28, in contrast, fails invoke a wake-up function and restarting the monitoring application. In some examples, beacon 18 may implement override criteria such that a beacon message directed to computing device 12A in area 28 may invoke a wake-up function, assuming certain override criteria can be satisfied.

An example authenticated area as defined herein may refer to a geographic area in which the presence of a patient device, e.g., computing device(s) 12, used for running the monitoring application implies the presence of patient 4. In FIG. 1, area 40 may represent an example authenticated area of the present disclosure. For patient 4, this geographic area 40 may be any known location for patient 4 that is likely to be more private than other areas, for instance, because access is restricted. In area 40, computing device 12A or computing device 12C can restart the monitoring application, while in view of patient 4, their caregiver, and/or clinician 26. Area 40 may be defined as a set of geographic coordinates within which a home/residence, retirement community, or an office or a place of business, as examples, provide a secure and authorized environment for administrating medical care for patient 4. Patient 4 may pre-select their home as an authenticated area via the monitoring application.

Area 28 may not be recognized as an authenticated area in some examples for a number of reasons. Given the clinical setting of area 28, it is visited by a substantial number of people in addition to patient 4 and is a workplace for a considerable number of employees. If one of patient 4's devices is in the clinic, it is not necessarily true that patient 4 will be found there. In one example, HMS 22 may override this classification and proceed to restart monitoring application if the override criteria are met.

Combined with network 16, HMS 22 enables computing device 12A or computing device 12C to use the monitoring application to electronically interrogate IMD 10 while patient 4 is at home, which may be represented in FIG. 1 as area 40, and then, relay the device interrogation data to HMS 22, for example, via computing device 12C. While patient 4 is in area 40 and without any patient involvement, a programmable beacon similar to beacon 18 may be effective in restarting the monitoring application, for instance, if the monitoring application stops running in computing device 12A. A beacon application running in computing device 12A may receive a beacon message from a beacon device in area 40 and grant authorization for automatically restarting the monitoring application.

Patient 4 may receive medical care while at a medical facility, such as a clinic of area 28 or hospital 38, but neither of those areas authenticate an automatic restart. If the monitoring application stops running in computing device 12A while patient 4 is in area 28, a programmable beacon device such as beacon 18 may not be effective in restarting the monitoring application. A beacon application running in computing device 12A may receive a beacon message and in turn, deny authorization for automatically restarting the monitoring application.

Certain conditions warrant authorizing an entity other than the patient to automatically restart the monitoring application. The present disclosure describes, as one example condition, the monitoring application being inoperative. To illustrate by way of example, while patient 4 is at home in area 40 or in a clinic of area 28, or otherwise has access to network 16, HMS 22 may receive (e.g., regular) uploads of patient data from device interrogations with IMD 10, which is only possible if the monitoring application is operative, for example, running in computing device(s) 12. After device interrogation, HMS 22 may initiate a remote review by a cardiologist and cannot do so unless the monitoring application is operative, for example, running in computing device(s) 12 and in a foreground according to an operating system.

HMS 22 may determine the monitoring application is inoperative through a variety of mechanisms, for example, based on a lack of communication from computing device(s) 12, for example, in response to a message (e.g., query, request, ping, and/or the like) from computing system(s) 20. HMS 22 may avail a second application running in computing device(s) 12 to provide status information regarding the monitoring application. If the second application returns status information indicative of an inoperative status, HMS 22 may, in turn, communicate a message (e.g., a beacon message) instructing the second application to perform a wake-up process, triggering execution of the monitoring application. There are a number of ways to perform the wake-up process of which a few examples utilize various hardware/software components (e.g., an operating system component) configured to start (e.g., restart) the monitoring application. For example, in response to the message invoking the wake-up process, the second application may communicate an inter-process message directed to the operating system component configured to allocate resources (e.g., in terms of processing power, storage capacity, network bandwidth, and/or the like) for one or more threads/processes and then, scheduling execution, by processing circuitry, of software code for the monitoring application, stored in memory, in the one or more threads/processes.

A beacon application associated with beacon 18 represents one example of the above second application and an alternative to the second application being a client application for HMS 22. In some examples, the beacon application determines that the monitoring application is inoperative and responsive to that determination, further determines whether a current location is in an authentication area as described herein. The beacon application may utilize input data (e.g., a beacon message) indicative of the current location received from beacon 18 (independent or instead of HMS 22 communicating a beacon message directed to computing device(s) 12). Beacon 18 may transmit the beacon message for invoking a wake-up function in computing device 12A, which is intended to prompt an operating system to execute the monitoring application. As described in further detail below, the beacon application may succeed in restarting the monitoring application if the current location corresponds to the authenticated area.

Computing device 12B may be incorporated into the apparel of patient 14, such as within clothing, shoes, eyeglasses, a watch or wristband, a hat, etc. In some examples, computing device 12B is a smartwatch or other accessory or peripheral for a smartphone computing device 12A. In some examples, wearable computing device 12B in the example illustrated by FIG. 1, may include electrodes and other sensors to sense physiological signals of patient 4, and may collect and store physiological information and detect episodes and other health events based on such signals.

In some examples, processing circuitry in a wearable device, e.g., wearable computing device 12B, or a laptop computer, e.g., personal computing device 12C, may execute same or similar logic as the logic executed by processing circuitry of computing device 12A. As an alternative, computing device 12C may be proprietary device provided by a manufacturer of IMD 10. In this manner, a wearable device, a laptop computing, and/or other device may perform some or all of the techniques described herein in the same manner described herein with respect to IMD 10. In some examples, the wearable device operates with IMD 10 and/or computing device 12A as potential providers of computing/storage resources and sensors for monitoring patient activity and other patient parameters. For example, the wearable device may communicate the patient data to computing device 12A or 12B, computing system 20A or 20B, and/or IoT devices 30A, 30B, or 30C for storage in non-volatile memory and for computing parameter values or metric values from patient physiological measurements and other sensed patient data.

Computing system 20B may be a local server device operating the computing service locally (e.g., via a radio access technology (RAT)). The monitoring application for IMD 10 and/or computing device 12A may configure one or both device(s) to expose function calls via an API available only on the local area network. In response to receiving input data from a device of computing system 20B, computing device 12A may identify an indication of a function call on that API and responsive to that identification, initiate execution of the monitoring application.

An entity, such a clinic of area 28, hospital 38, or a retirement community of area 40, may operate a computing system, such as computing device 12C of area 40 or computing system 20A of area 28, as a resource in their provision of medical care for patient 4 via HMS 22. Computing device 12C may include processing circuitry configured with logic that, when executed, is operative to detect a presence of a patient device, such as computing device 12A, within area 40 based on a broadcasted message (e.g., a heartbeat) distributed by that patient device. In one example assuming the broadcasted message also indicates an operating status of the monitoring application, computing device 12C may return a beacon message to restart the monitoring application if that application becomes inoperative.

Computing device 12C may avail IoT device 30B for automatically restarting the monitoring application according to other examples. To illustrate at least one, IoT device 30B may detect a presence of patient 4 and/or their patient device in area 40 based on broadcasted messages from computing device 12A. Computing device 12C may configure IoT device 30B to return beacon messages similar to the above-mentioned example. Furthermore, IoT device 30B may be configured to return a beacon message in response to a broadcasted message from computing device 12A even if the broadcasted message does not indicate an inoperative monitoring application. As an alternative, computing device 12C may configure IoT device 30B to broadcast beacon messages to nearby devices to ensure every patient device within area 40 has an operative monitoring application running for their corresponding patient. As another alternative, computing device 12C may configure IoT device 30B to broadcast location data to a beacon application running in computing device 12A.

In general, the monitoring application is configured to initiate health event monitoring of various types of information, for example, by identifying, in a dataset for patient 4, indicia of interest regarding health (e.g., cardiac health) of patient 4. The monitoring application may be a client application for HMS 22. The monitoring application, once restarted, is once again capable of communicatively coupling to HMS 22. The second application in computing device 12B may be different from the monitoring application. In other examples, without computing device 12A executing the monitoring application, the application logic may be executed to run in a background or foreground of computing device 12B as the monitoring application.

For the monitoring application running in computing device 12A, a second and different application or process (e.g., an operating system process, a beacon application, or a client application for HMS 22) running in computing device 12A may implement an API with functionality for restarting the monitoring application, for example, by way of a function call on that API. There are a number of mechanisms in computing device 12A that are configured to invoke such a function call, causing execution of appropriate monitoring application logic (e.g., software code) and/or activating components of that logic for performing one or more operations including a boot script or a data submission to HMS 22. The API may implement a callback method (e.g., run ()) configured to execute the boot script for the monitoring application. When the second application receives a command (e.g., in a message payload) invoking the function call to perform the monitoring application startup process, the API passes the callback method to the operating system interface for running applications. The operating system interface instantiates the monitoring application context and then, executes the boot script. There are a number of example techniques for initiating any type of data submission, such as, for example, by configuring (application) logic with functionality that when invoked, triggers an automatic transmission (e.g., broadcast) by computing device 12A of output data to one or more receiving devices.

HMS 22 may be configured to use additional technologies to restart the monitoring application and perform additional operations. HMS 22 may use BLE to remotely trigger execution and, possibly, a medical device interrogation by computing device(s) 12. HMS 22 may use BLE to remotely set a desired operating mode, such as a normal operating mode, for the monitoring application. While the normal operating mode is active, IMD 10 and/or computing device 12A may be programmed to upload datasets of patient data in accordance with a schedule. HMS 22 may use BLE to cause further reprogramming of computing device 12A to change an operating mode, for example, to the modify the schedule. HMS 22 may leverage BLE hardware/software at computing device(s) 12, IoTs 30, and computing system(s) 20 to accomplish the restart and any additional operation, for example, by sending a beacon message to computing device(s) 12 from IoTs 30, computing system(s) 20, and other devices in the authenticated area.

The medical systems and techniques create hardware/software components to improve the efficiency of assessing patients with implantable medical devices. Some medical systems and techniques allow certain devices to control the monitoring application without a human user. By removing human error, HMS 22 is able to keep a connection open to receive recent patient data including health event alerts. Hence, system 2 allows clinics to keep up with increasing demands for patient assessment and/or device interrogation while maintaining/improving patient safety (by accurate diagnosis of arrhythmias and device abnormalities) and convenience (by reducing or eliminating the number of trips to physician's offices).

IMD 10 may generate a health event alert (e.g., including an audible and/or tactile alarm to get the immediate attention of patient 4 or clinician 26 of a potential health event) and at a later point-in-time, receive, from computing system 20A, a message directing IMD 10 to perform an action. In one example, computing system 20A may communicate the message to computing device 12A (e.g., via IoT device 30A, IoT device 30B, and/or another intermediate device) causing an application running in IMD 10 to output datasets of patient data corresponding to the potential health event. An intermediate device such IoT device 30A may operate as an agent of and as directed by HMS 22, or may be independent of HMS 22 but still operative as an intermediary for messages directing IMD 10 to perform an action.

HMS 22 may determine that a potential health event is a false positive and communicate a message instructing IMD 10 to silence or cancel an alarm (e.g., the audible and/or tactile alarm) for the potential health event. There may be cancellation criterion for the potential health event to satisfy in order to qualify the alarm for cancellation. Instead of or in addition to the alarm, IMD 10 may broadcast an alert message to any receiver within a certain proximity upon detecting the potential health event. The detection of the potential health event by IMD 10 may be in response to results based on an analysis of current patient data (e.g., recently sensed physiological signals of patient 4). HMS 22 may predict an alert by computing device 12A and/or an alarm by IMD 10 corresponds to a false detection and then, communicate a message instructing IMD 10 to cancel the alert and/or the alarm.

While computing device(s) 12 may be communicatively coupled to IMD 10, it should be noted several alternatives to IMD 10 including multiple medical devices or no medical device. In some examples, patient 4 may have computing device 12 and IMD 10 as part of independent systems, and in at least example, patient 4 does not have a medical device implanted into their body. Because the patient's mobile device may allocate some of its resources for use with the patient's medical device, the medical system and the techniques described herein may configure the mobile device and the medical device to cooperate during medical device interrogation or patient data assessment, for instance, by operating on a same layer to the health monitoring service.

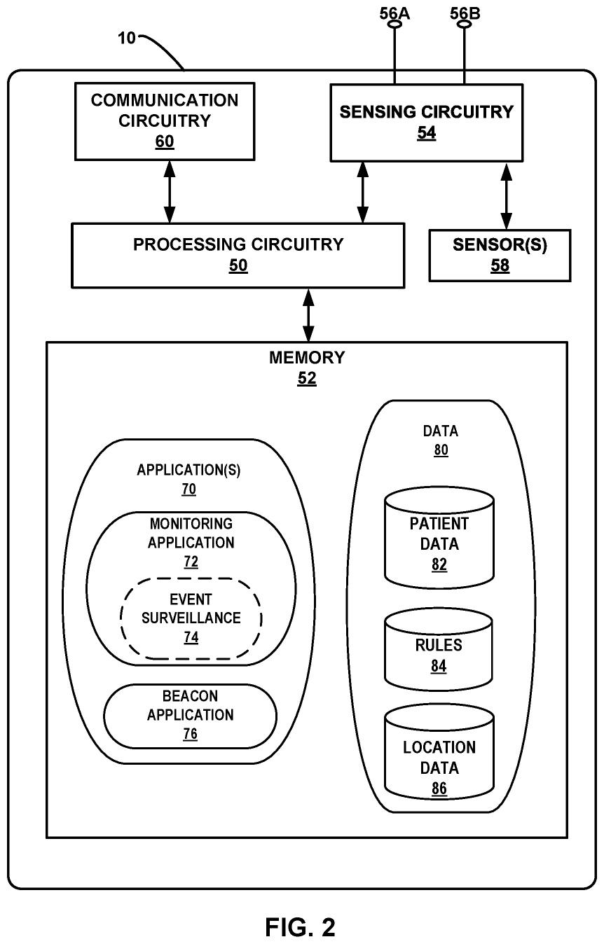

FIG. 2 is a block diagram illustrating an example configuration of IMD 10 of FIG. 1. As shown in FIG. 2, IMD 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may monitor signals from electrodes 56 in order to, for example, monitor electrical activity of a heart of patient 4 and produce ECG data for patient 4. In some examples, processing circuitry 50 may identify features of the sensed ECG, such as heart rate, heart rate variability, intra-beat intervals, and/or ECG morphologic features, to detect an episode of cardiac arrhythmia of patient 4. Processing circuitry 50 may store the digitized ECG and features of the ECG used to detect the arrhythmia episode in memory 52 as episode data for the detected arrhythmia episode.

In some examples, sensing circuitry 54 measures impedance, e.g., of tissue proximate to IMD 10, via electrodes 56. The measured impedance may vary based on respiration and a degree of perfusion or edema. Processing circuitry 50 may determine physiological data relating to respiration, perfusion, and/or edema based on the measured impedance.

In some examples, IMD 10 includes one or more sensors 58, such as one or more accelerometers, microphones, optical sensors, temperature sensors, pressure sensors, and/or any other sensors. In some examples, sensing circuitry 52 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52.

Memory 52 may store applications 70 executable by processing circuitry 50, and data 80. Applications 70 may include a monitoring application 72 and beacon application 76. Processing circuitry 50 may execute monitoring application 72, invoking event surveillance 74, to detect a health event of patient 4 based on combination of one or more of the types of physiological data described herein, which may be stored as patient data 82. In some examples, patient data 82 may additionally include data sensed by other devices, e.g., computing device(s) 12, and received via communication circuitry 60. Event surveillance 74 may be configured to operate with monitoring application 72 to apply rules 84 to patient data 82. Rules 84 may include one or more models, algorithms, decision trees, and/or thresholds. In some cases, rules 84 may be developed based on machine learning.

Processing circuitry 50 may execute monitoring application 72 and in accordance with logic of the executed application, perform a number of operations. In some examples, in response to an indication of the current patient location, processing circuitry 50 transmits, via communication circuitry 60, the datasets of patient data 82 to computing device(s) 12 and/or computing system(s) 20 of FIG. 1. This transmission may be included in a message indicating the health event, as described herein. Transmission of the message may occur on an ad hoc basis and as quickly as possible as the response to the indication. Communication circuitry 60 may include any suitable hardware, firmware, software, or any combination thereof for wirelessly communicating with another device, such as computing devices 12 and/or IoT devices 30.

Some embodiments of the medical system couple IMD 10 with a remote computing system configured to run one or more computing services including a health monitoring (computing) service. The remote computing system (e.g., computing system(s) 20 of FIG. 1) may communicate packetized data to IMD 10 located within a geofenced geographic area, prompting IMD 10 to execute program code that implements an operation of interest. That packetized data may comprise a message in which the message data identifies the operation of interest. In some examples, the health monitoring service may classify a particular operation as an operation of interest. For instance, operation results may provide information beneficial to patient 4. The health monitoring service may desire the operation results, for example, to improve upon the medical care provided patient 4. HMS 22 of FIG. 1, as an example of the health monitoring service, may be configured to trigger restarting monitoring application 72 by beacon application 76. Once triggered, automated data submissions may be based on certain conditions such as a state of operation, recency of stored patient data, other functionality of the service, and so forth.

The health monitoring service may invoke functionality to perform an example action that when activated by an indication of current patient location, triggers an execution of the monitoring application based on its operating status. While in operation, IMD 10 uploads one or more datasets of patient data, but, at times, IMD 10 fails to successfully upload any data. When IMD 10 does not perform a data upload within an expected time frame, IMD 10 may be determined to be inoperative.

Monitoring application 72 may be configured to submit various datasets of patient data 82, in some examples, as a response to being prompted by communications (e.g., messages) from a device of the health monitoring service, such as HMS 22 of FIG. 1, a patient device, such as computing device 12A of FIG. 1, and/or a beacon device, such as IoT device 30 of FIG. 1. A message may indicate a current patient location and if that location corresponds to a geofenced area, beacon application 76 is configured to automatically restart monitoring application 72, for example, by initiating a function call to have logic for monitoring application 72 executed (e.g., an operating system) and then, run as a foreground process. This may be performed in response to requests from a local device (e.g., a beacon device) for the health monitoring service (e.g., HMS 22). As an option, beacon application 76 may be executed in IMD 10 to run as a client or an agent of the health monitoring service.

The health monitoring service may initiate an action that causes beacon application 76 to perform some functionality with monitoring application 72. IMD 10 may receive from the device of the health monitoring service, the patient device, and/or the beacon device a message identifying beacon application 76 as a recipient. Based on contents of the message, beacon application 76 may be directed to transmit an inter-process communication invoking an appropriate function call on monitoring application 72. Receiving the communication may cause monitoring application 72 to perform the initiated action of the health monitoring service. In one example, the function call may be configured to open/start or restart monitoring application 72. In another example, the function call may be configured to transition monitoring application 72 from a sleep state or an active state. In yet another example, the function call may be configured to resume stalled, terminated, and/or dormant operations of monitoring application 72.

In some examples where monitoring application 72 is configured to receive communications over the network from the health monitoring service, monitoring application 72 may be running in a background and in response to at least one communication, transition to running in a foreground. If monitoring application 72 is not running, some communications initiate execution of monitoring application 72. When monitoring application 72 starts running in the foreground after being restarted, IMD 10 may automatically complete a data submission (e.g., expected by HMS 22 of FIG. 1 such as a pending transmission of device interrogation data). The logic for monitoring application 72 may leverage the network connectivity with the computing system of the health monitoring service and transmit messages (e.g., a response message to a query) in packetized data.

The activation may be triggered by beacon application 76 receiving an indication of patient 4 entering the geofenced area. The health monitoring service may facilitate other forms of activation (without any involvement from patient 4) as set forth herein.

As examples, event surveillance 74 may be configured to detect cardiac arrhythmias, worsening heart failure, or other cardiac health events (or simply “cardiac events”) based on an EGM/ECG recording cardiac activity data and/or various physiological parameters indicative the electrical or mechanical activity of heart 6 of patient 4 (FIG. 1). In some examples, event surveillance 74 may detect stroke based on the cardiac activity data. In some examples, sensing circuitry 54 may detect brain activity data, e.g., an electroencephalogram (EEG) via electrodes 56, and event surveillance 74 may detect stroke or a seizure based on the brain activity alone, or in combination with cardiac activity data or other physiological data. When event surveillance 74 detects a health event, event surveillance 74 may store patient 82 comprising sensed information that led to the detection (and in some cases a window of data preceding and/or following the detection). Prior to transmitting patient data 82, monitoring application 72 may apply an adjudication technique to either confirm or reject the detected health event. Unless rejected by the adjudication technique, monitoring application 72 may generate an alarm for patient 4 or an alert for the health monitoring service.

The medical system and techniques of the present disclosure may package IMD 10 with external computing resources that results in enhancing IMD 10 in a number of ways. The medical system and techniques descried for FIG. 1 includes external locations with additional storage resources for patient data. The medical system and techniques described for FIG. 1 benefit from engaging (remote) computing services launched by a remote computing system instead of consuming local processing resources for new/updated software components. These capabilities may be new capabilities, firmware updates, support software and other components to improve the operations of IMD 10.

An external device for IMD 10 may include software/hardware components that provide software applications running in IMD 10 with access to storage, processing, and network resources. IMD 10 may use the external device for enhancing functionality of monitoring application 72 running in the external device. When the external device is a mobile device, a mobile application may be configured to facilitate IMD 10 interrogation and patient data assessment process more efficient. By implementing the techniques of the present disclosure, the external device may improve upon the efficiencies of device interrogation and patient data assessment.

Another example external device may be a beacon device, which may be implemented as an IoT device (e.g., IoT device 30 of FIG. 1). The beacon device may be attached to any physical object and positioned throughout a geofenced area. Via the beacon device, beacon application 76 may be configured to receive beacon communications from the health monitoring service or and/or a server of a local area network. In some examples, the beacon communications are messages, similar to those described herein, that include location data 86 (e.g., an indication of a current patient location). Some message prompt an invocation of functionality provided by beacon application 76. As directed in an example function call, beacon application 76 may communicate to monitoring application 72 (e.g., as an inter-process communication) based on the message received from the beacon device.

Monitoring application 72 may become inoperative after running in the background of an operating system of IMD 10. In such instances, beacon application 76 has to initiate execution of monitoring application 72 and/or transition the executed logic of monitoring application 72 from operating in the background to the foreground. If monitoring application 72 is running in the background, for instance, in sleep mode, beacon application 76 may run a wake-up process with the operating system of IMD 10 to transition monitoring application 72 to the foreground.

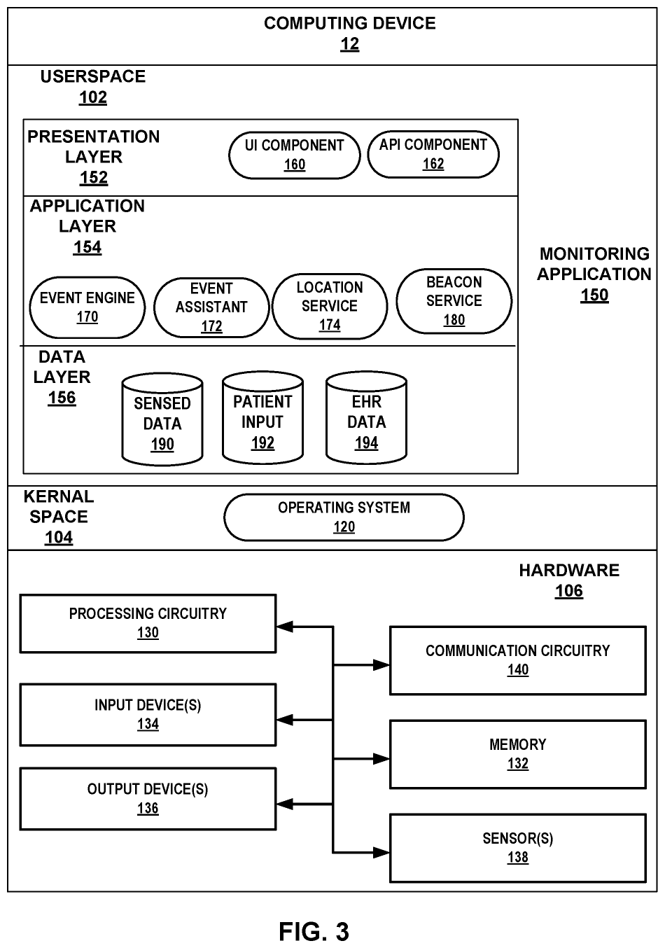

FIG. 3 is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a laptop, a tablet computer, a personal digital assistant (PDA), a smartwatch or other wearable computing device. In some examples, IoT devices 30 may be configured similarly to the configuration of computing device 12 illustrated in FIG. 3.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, one or more sensors 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random access memories (DRAM), ferroelectric random access memories (FRAM), static random access memories (SRAM), and other forms of volatile memories known in the art. Memory 132, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, audio, and visual output. Output devices 134 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

One or more sensors 138 of computing device 12 may sense physiological parameters or signals of patient 4. Sensor(s) 138 may include electrodes, accelerometers, an optical sensor, and other sensors, and sensing circuitry (e.g., including an ADC), similar to those described above with respect to IMD 10 and FIG. 2.

Communication circuitry 140 of computing device 12 may communicate with other devices by transmitting and receiving data. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, Wi-Fi (e.g., 802.11 or 802.15 ZigBee), Bluetooth®, or BLE.

As shown in FIG. 3, monitoring application 150 executes in user space 102 of computing device 12. Monitoring application 150 may be logically divided into presentation layer 152, application layer 154, and data layer 156. Presentation layer 152 may include a user interface (UI) component 160, which generates and renders UI views of health monitoring application 150. Presentation layer 152 may include API component 162, which instantiates an API with functionality for using software modules of application layer 154.

Application layer 154 may include, but is not limited to, an event engine 170, event assistant 172, location service (client) 174, and beacon service (client) 180. Event engine 172 may be responsive to receipt of an indication of a current patient location from a device of HMS 22 or from a different device. Event engine 172 may control performance of any of the operations in response to the indication of the current patient location, particularly, if the current patient location indicates patient 4 entered a geographic area (e.g., geofenced area). Event engine 172 may be responsive to receipt of a transmission from IMD 10 indicating that IMD 10 detected a health event, e.g., an acute health event.

In some examples, event engine 170 analyzes sensed data 190, and in some examples, patient input 192 and/or EHR data 194, to determine whether there is a sufficient likelihood that patient 4 is experiencing the health event. Sensed data 190 may include data received from IMD 10 as part of a transmission, additional data transmitted from IMD 10, e.g., in “real-time,” and physiological and other data related to the condition of patient 4 collected by computing device(s) 12 and/or IoT devices 30. As examples, sensed data 190 from computing device(s) 12 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

A computing system for operating a health monitoring service, HMS 22, may be configured to communicate, over a network, a request for patient input 192 as an example data submission. Health monitoring application 150 may receive the request, generally, in the form of some content for presentation in a user interface (UI) view. In one example, health monitoring application 150 generates the UI view with UI elements configured to receive patient input 192.

Patient input 192 may include responses to queries posed by health monitoring application 150 regarding a current condition of patient 4, input by patient 4 or another user, such as clinician 26. The queries and responses may occur responsive to entering a geofenced area and receiving a message from a device of HMS 22, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition, symptoms, or treatments of patient 4 described above. EHR data 194 may relate to history of cardiac arrest, tachyarrhythmia, myocardial infarction, stroke, seizure, chronic obstructive pulmonary disease (COPD), renal dysfunction, or hypertension, history of procedures, such as ablation or cardioversion, and healthcare utilization. EHR data 194 may also include demographic and other information of patient 4, such as age, gender, height, weight, and BMI.

An assistant 172 may provide a conversational interface for patient 4 and/or clinician 26 to exchange information with computing device 12. Event assistant 176 may query the user regarding the condition of patient 4. Responses from the user may be included as patient input 192. Event assistant 172 may use natural language processing and context data to interpret utterances by the user. In some examples, in addition to receiving responses to queries posed by the assistant, assistant 172 may be configured to respond to queries posed by the user. In some examples, event assistant 172 may provide directions to and respond to queries regarding treatment of patient 4 from patient 4 or clinician 26.

Location service 174 may determine the location of computing device 12 and, thereby, the presumed location of patient 4. Location service 174 may use global position system (GPS) data, multilateration, and/or any other known techniques for locating computing devices.

Beacon service 180 may receive messages from a (local or remote) health monitoring service running in a computing system. The health monitoring service may prompt computing device 12 to restart, via an operating system, monitoring application 150 and then, via monitoring application 150, to perform one or more operations. One operation may be a data submission, for example, a missed transaction that failed to complete for some reason (e.g., being shutdown).

In further response to the indication of patient 4's current location, monitoring application 150 may configure computing device 12 for synchronizing sensed patient data and device interrogation data with an external storage location. The patient is a user of a medical device, such as IMD 10 of FIG. 1, which generated the sensed patient data from various signals. In some examples, rules engine 172 may store in memory for event assistant 172 information defining a schedule for clinic visits and services from the computing system of HMS 22.

API component 162 may include functionality that when invoked, performs a desired operation. A computing service, such as a health monitoring service (e.g., HMS 22 of FIG. 1) or a location service, may communicate a message to initiate the desired operation. In one example, the computing system of the health monitoring service may communicate a message directed to API component 162. The message may identify a function call, for example, for executing (e.g., restarting) monitoring application 150 if that application becomes inoperative.

A variety of actions are enabled for the patient device(s) upon recognizing events including non-health events as described herein. HMS 22 may initiate an action that benefits the patient's health monitoring (e.g., by restarting monitoring application 150. A computing device under direction of HMS 22 may communicate signal data that ultimately, causes event engine 172 to perform one or more operations. In some examples, monitoring application 150 may become inoperative; however, the health monitoring service may continue to require/desire recent patient data from the patient device(s) and for that purpose, perform one or more operations to trigger a restart of monitoring application 150.

The medical systems and techniques may implement location-based and remote control technologies to enable such a restart in computing device(s) 12, such as in response to a determination that a current location of the patient and/or computing device(s) 12 corresponds to an authenticated area and to the determination that the monitoring application 150 is inoperative. There are a number of ways for

Location data (e.g., geographic coordinates) for the geographic area on which the patient resides may be programmed into a computing device associated with HMS 22 such that the patient entering the geographic area (e.g., their home or residence) triggers the restart of monitoring application 150. The computing device may determine the patient is currently located within the geographic area through a variety of mechanisms, and communicate a message (e.g., an indication of a current patient location); one example mechanism may enable the computing device to recognize the patient device (or only the patient) within a close proximity, for example, by broadcasting signal data to illicit a response and/or receiving (e.g. a broadcast of) signal data from the patient device. Detecting the patient device through such a mechanism causes the computing device to trigger a restart of monitoring application 150 at the patient device. Detecting the patient may be optional in some examples; for instance, the computing device may broadcast signal data that when captured by the patient device, causes the patient device to initiate the restart of monitoring application 150. Radio waves may embody the signal data for the indication. Hence, a geofence can be configured for the home or residence using geographic coordinates.

The medical systems and techniques may implement geofencing technology to enable remote restarting of any application, such as when the patient is at their residence and without their knowledge, monitoring application 150 becomes inoperative. A device (e.g., a beacon device) within a geofenced area (such as the above mentioned geographic area) may operate as part of a local health monitoring service running on a local computing system such that the patient entering their residence causes the beacon device to detect a presence of the patient, recognize an event indicating an inoperative monitoring application 150, and triggering a restart of monitoring application 150 from computing device 12.

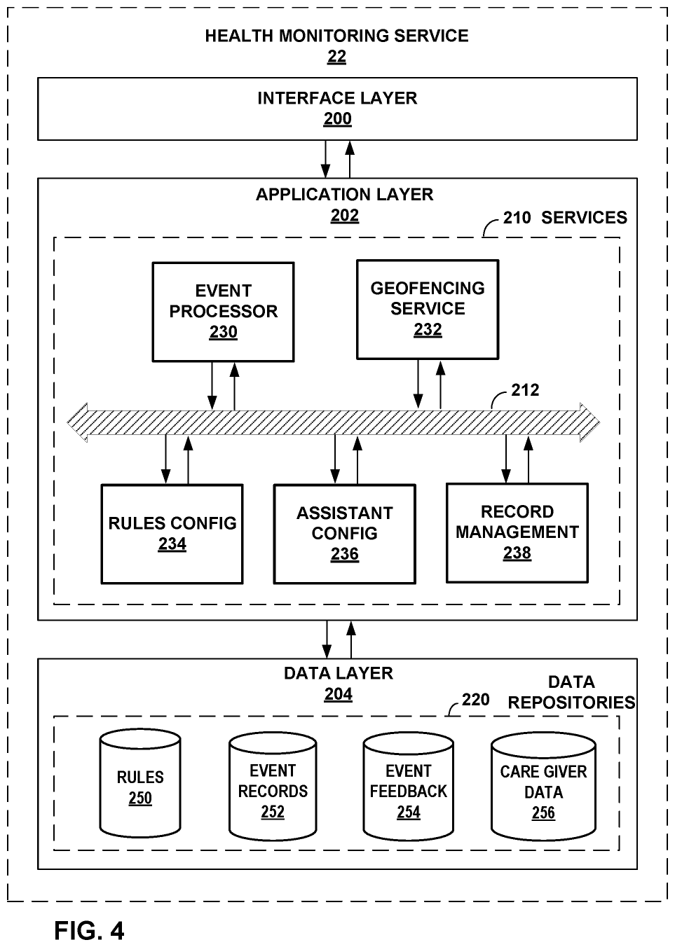

FIG. 4 is a block diagram illustrating an operating perspective of HMS 22. HMS 22 may be implemented in a computing system 20, which may include hardware components such as those of computing device 12, embodied in one or more physical devices. FIG. 4 provides an operating perspective of HMS 22 when hosted as a cloud-based platform. In the example of FIG. 4, components of HMS 22 are arranged according to multiple logical layers that implement the techniques of this disclosure. Each layer may be implemented by one or more modules comprised of hardware, software, or a combination of hardware and software.

Computing devices, such as computing devices 12, IoT devices 30, computing devices 38, and computing device 42, operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 4, HMS 22 also includes an application layer 202 that represents a collection of services 210 for implementing the functionality ascribed to HMS herein. Application layer 202 receives information from client applications, e.g., an alert of an acute health event from a computing device 12 or IoT device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnections or set of interfaces that allows different services 210 to send messages to other services, such as by a publish/subscription communication model.

Data layer 204 of HMS 22 provides persistence for information in PPEMS 6 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of services 230-238 is implemented in a modular form within HMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads or software instructions generally for execution on one or more physical processors.

Event processor service 230 may be responsive to receipt of an alert transmission from computing device(s) 12 and/or IoT device(s) 30 indicating that IMD 10 detected an acute health event of patient and, in some examples, that the transmitting device confirmed the detection. Event processor service 230 may initiate performance of any of the operations in response to detection of an acute health event ascribed herein to HMS 22, such as communicating with patient 4, clinician 26, and care providers 40, activating drone 46 and, in some cases, analyzing data to confirm or override the detection of the health event by IMD 10 and cancel any alarm or alert generated for the heath event.

Record management service 238 may store the patient data included in a received alert message within event records 252. Alert service 232 may package the some or all of the data from the event record, in some cases with additional information as described herein, into one more alert messages for transmission to clinician 26 and/or care providers 40. Care giver data 256 may store data used by alert service 232 to identify to whom to send alerts based on locations of potential bystanders 26 and care givers 40 relative to a location of patient 4 and/or applicability of the care provided by care givers 40 to the acute health event experienced by patient 4.

In examples in which HMS 22 performs an analysis to confirm or override the detection of the health event by IMD 10, event processor service 230 may apply one or more rules 250 to the data received in the alert message, e.g., to feature vectors derived by event processor service 230 from the data. Rules 250 may include one or more models, algorithms, decision trees, and/or thresholds, which may be developed by rules configuration service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, the Apriori algorithm, K-Means Clustering, k-Nearest Neighbor (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, in addition to rules used by HMS 22 to confirm health event detection, (or in examples in which HMS 22 does not confirm event detection) rules 250 maintained by HMS 22 may include rules 196 utilized by computing devices 12 and rules 84 used by IMD 10. In such examples, rules configuration service 250 may be configured to develop and maintain rules 196 and rules 84. Rules configuration service 234 may be configured to modify these rules based on event feedback data 254 that indicates whether the detections and confirmations of acute health events by IMD 10, computing device 12, and/or HMS 22 were accurate. Event feedback 254 may be received from patient 4, e.g., via computing device(s) 12, or from care providers 40 and/or EHR 24. In some examples, rules configuration service 234 may utilize event records from true and false detections (as indicated by event feedback data 254) and confirmations for supervised machine learning to further train models included as part of rules 250.

As illustrated in the example of FIG. 4, services 210 may also include an assistant configuration service 236 for configuring and interacting with event assistant 176 implemented in computing device 12 or other computing devices.

In some examples, health monitoring service 250 may be active on a different network or a same network as a patient who also is a medical device user. To operate within a geographic area, health monitoring service 250 configures one or more computing devices to be (e.g., network) accessible to the patient's devices, including the medical device and possibly their mobile device(s). The patient may enter the (e.g., geofenced) geographic area and obtain (e.g., instantaneous) access to a computing device (and if needed, to health monitoring service 250) over at least one connection.

The present disclosure describes herein a number of applicable technologies for a connection between the patient's device(s) and a device of health monitoring service 250, including radio technologies enabling data communication between devices in the form of radio waves. The present disclosure generally describes signal data when referencing radio waves encoding data. The patient may have a mobile device configured to communicate, in the form of signal data, various information over any compatible connection (e.g., a wireless connection over NFC, Bluetooth, or BLE; a network connection over a local area network or the Internet; and so forth). The patient's mobile device may be configured to capture signal data from a device located in the geographic area and as a response, initiate some action to restart monitoring application 150 after becoming inoperable. Such a connection may be further characterized as a local connection with a local device, such as a beacon device, a device with a GPS module (e.g., transmitted and/or receiver), a local computing device operating as directed by health monitoring service 250, a medical device, and any other device compatible with the local connection. If appropriate, the medical device may be configured to initiate a restart of monitoring application 150 upon determining that the application became inoperable.

In general, the above-mentioned signal data may be generated to convey any type of information including location data. The computing device (e.g., under direction of a computing service such as health monitoring service 250) may communicate signal data comprising an indication (e.g., message) of a (e.g., current) patient location. The computing device may support other messages (e.g., control messages) including commands/directives to restart an inoperative application, interface messages invoking function calls for restarting an inoperative application, and so forth.

As an alternative computing service to heath monitoring service 250, the patient's mobile device may receive messages (e.g., an indication of current patient location) from a location service (e.g., a GPS service). A computing device associated with the location service may be configured within the geographic area of the patient. According to another alternative example, the patient's mobile device may be configured (e.g., with a GPS receiver) for receiving, from the computing service directly and without the computing device (e.g., operating on behalf of health monitoring service 250 or the location service), location data indicative of a current patient location.

Medical systems and techniques described herein are applicable to computing systems for personal health monitoring of patients without actually being present in a same area and away from a patient's location (i.e., remotely). In summary, medical systems and techniques described herein are capable of reducing the time it takes for health monitoring service 250 to interrogate an implantable medical device, such as an implantable pacemaker, ventricular assist device (VAD), defibrillator, cardiac monitor, or other cardiac device. Improved efficiency and patient flow can be observed with this technology in addition to improved input from device specialists when the patients are remotely located and improved clinical decision making because of the prompt input from specialists.

Alternatively, a geographic area may include a local health monitoring service 250 configured with a GPS receiver operative to receive coordinates of the patient at their current location and/or coordinates of the clinic. Based on a comparison between both sets of coordinates, a device of the local health monitoring service may determine when the patient is currently located in the clinic and then, responsive to the determination, initiate any data submission that is either past due or due at a clinic appointment time or a current patient time. A different embodiment of the location data may be an indication (e.g., a message) that a current patient location is within a pre-defined proximity (e.g., an authenticated area).

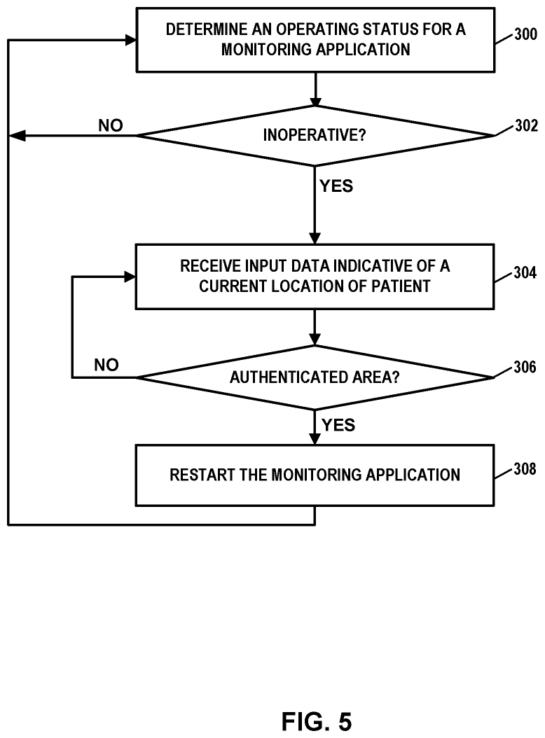

FIG. 5 is a flow diagram illustrating an example operation by a computing device of a medical system that operates in accordance with one or more techniques of the present disclosure. The example operation depicted in FIG. 5 is described with respect to a computing device 12 depicted in FIGS. 1 and 3, but may be described with respect to any one or more devices, e.g., computing devices 12, computing systems 20, IoT devices 30, IMD 10, or health monitoring service (HMS) 22 of FIGS. 1-4 that may implement a health monitoring service and/or client applications of the service. The techniques described herein may be performed by processing circuitry of any one or more of these devices.

According to the illustrated example of FIG. 5, processing circuitry of system 2 (e.g., processing circuitry 130 of one or more computing device(s) 12) provides a computing service for health event monitoring based on a patient's physiological data (e.g., sensed data 190) generated by sensing circuitry 52 of IMD 10 and patient input (e.g., patient input 192). According to the illustrated example of FIG. 5, the processing circuitry detects changes in the patient's health caused by an acute health event such as an arrhythmia or a cardiac arrest.

The present disclosure describes “health monitoring” as a computing service engaging one or more devices to support any given patient with a medical device such as an implantable medical device or a wearable device. Such a patient is likely to benefit from a health monitoring service that expands a patient's present medical care, for instance, beyond the patient's personal devices.

As described above, patients typically rely upon personal devices (e.g., medical devices and mobile devices) for health monitoring outside of a clinical setting. When limited to the patient's personal device(s), the patient cannot benefit from external resources. A patient may accept health monitoring to some degree beyond their (personal) device, such as from an external device over a network; however, the patient's own personal device inhibits such a manner of health monitoring by causing delays in medical device interrogation, inconsistencies in patient data, inefficiencies in patient data assessment and so forth. An external device may be communicatively coupled to the patient's personal device(s) and, via a number of mechanisms, arrange for submissions of patient data and other transmissions from the patient device(s) to either the external device or to another external location.

As described herein, a health monitoring service, in general, detects changes in the health of patients based upon patient data. For example, by way of the above-mentioned patient engagement, the computing system running the health monitoring service may perform an assessment of any patient with as pacemakers, defibrillators, monitors, and other examples of implantable medical device(s) (e.g., IMD 10 of FIG. 1). The health monitoring service may configure the computing system to operate as a beacon device and advantageously employ beacon messages to trigger execution and/or invocation of application logic on the patient device.

To commence the example operation of FIG. 5, the processing circuitry determines an operating status for a monitoring application in the patient device (300). The patient device may be a mobile device configured to receive beacon messages and in response, invoke appropriate functionality for each corresponding application. As described herein, the monitoring application may be configured to run in the mobile device as a client application for the health monitoring service and to that end, performs various functionality on behalf of the health monitoring service and to benefit the patient. The monitoring application provides the computing system with various patient data generated by the medical device.

In the example of FIG. 5, the processing circuitry determines whether the monitoring application is operative in the patient device or is to be considered inoperative (302). The patient device may provide an operating status of the monitoring application to another, second application in a number of ways. The second application may run in a same computing device or a different computing device. An operating system may generate the operating status as part of a diagnostic tool. It should be noted that there are alternatives to using the diagnostic tool for determining whether the monitoring application is operative or inoperative and therefore, the diagnostic tool is not necessary for the determination; for example, if the monitoring application fails to transmit any patient data to the health monitoring service despite being requested and/or scheduled to do so or fails to respond to a recent message from the health monitoring service, the processing circuitry may determine that the monitoring application has stopped and become inoperative.

In response to determining the monitoring application is not inoperative (and therefore, operative) (NO of 302), the processing circuitry may return to determining the operating status of the monitoring application and wait for the operating status to change from operative to inoperative (300).

In response to determining the monitoring application is inoperative (YES of 302), the processing circuitry receives input data indicative of a current location of a patient (304). In one example, the input data may be an indication message directed to a second application for initiating a function call configured to restart a monitoring application based on that patient being located in a geofenced geographic area authenticated as a known patient location (e.g., a home or residence). The indication may be a set of GPS coordinates provided by a GPS receiver in a computing device located within a certain proximity of one of the patient device(s). The patient device may be configured with the GPS receiver for receiving the set of GPS coordinates from the computing device or, alternatively, directly from a location service such as a GPS service. In some examples, a local computing system may run the location service for the (e.g., geofenced) geographic area and communicate a message to the patient device in response to the patient's presence in that area. As an alternative, the input data may notify the patient of their proximity to the geographic area. The message may be arranged into packetized data in accordance with any communication technology (e.g., protocol).

As described herein, an example remote health monitoring service 250 may be configured as a computing service (e.g., a cloud service) that is accessible over a network from any geographic area using any number of communication protocols as described herein such as the Internet. Remote health monitoring service 250 may combine local health monitoring service 250 with a network service (e.g., a cloud service) running on a network-accessible resource and built using network functions that are compatible with any number of protocols. The network-accessible resource at the external location may be used to store patient data and facilitate the patient's medical care. Similar to local health monitoring service 250, the remote health monitoring service 250 may initiate a main process of the monitoring application to performs a number of operations including automatically restarting an inoperative application.

The computing system of the geofenced geographic area may avail a low energy wireless communication technology, such as BLE, as the underlying protocol for communicating a message indicative of the current location of a patient. In some examples, the indication may be in the form of a beacon message (e.g., advertisement) that a BLE-enabled beacon device broadcasts at a regular interval. The computing system may operate as a beacon device and/or configure one or more beacon devices to operate within the geofenced geographic area.

The patient device, by capturing signal data (e.g., radio waves) of a wireless connection a beacon device, receives the beacon message and based on message content, proceeds to determine that their current location is within the geofenced geographic area. The patient device may determine that the beacon message is directed towards an application and configured to trigger actions by the patient device. In one example, the computing system of the geofenced geographic area, operating as an iBeacon™ device in accordance with Apple® iBeacon™ technology, may communicate an iBeacon™ advertisement message, as one example embodiment of the beacon message, to any device in that area. In turn, an iBeacon™ application running on the patient device, while listening for signals from iBeacon™ devices, senses the iBeacon™ device operated by the computing system by capturing the iBeacon™ advertisement message. In response to the advertisement, the iBeacon™ application communicates an acknowledgement of a connection with the computing system running the computing service for the geofenced geographic area. The connection with the computing system allows the computing service to provide secure and hyper-contextual content (e.g., formal documents). As an alternative to iBeacon™ technology, the patient device may employ Eddystone™ technology also receive/transmit beacon messages and

In the example of FIG. 5, the processing circuitry determines whether the patient is within an authenticated (e.g., geofenced) geographic area (e.g., area 40 of FIG. 1) based on the current location of the patient's device (306). Based on a determination that the patient and/or the patient's device is not currently located in the authenticated area (NO of 306), the processing circuitry may wait for a change to the current location such as when the patient moves to a second geographic area and the patient device receives new input data indicative of the second geographic area as a new current location (304). In one example, a beacon application running in the patient device(s) may recognize the second geographic area as an environment in which the patient resides, authorizing the beacon application to trigger execution of the monitoring application. A connection with the computing system running the health monitoring service further facilitates execution of logic for the monitoring application and access to a patient account (e.g., without first providing a credential).

In the example of FIG. 5, based on a determination that the patient and/or the patient's device is currently located in the authenticated area (YES of 306), the processing circuitry restarts the monitoring application (308), for example, via a computerized process invoked by way of a function call. An example embodiment of the function call may be a wake up function initiated through an API. As described herein, the API may be implemented by a second application known as a beacon application. Typically, without patient involvement, the processing circuitry of the patient device may be unable to restart the monitoring application, for example, to initiate an interrogation of a medical device and possibly, additional actions. Having an API to implement functionality for the health computing service to use allows another computing device (and another application) to initiate function calls for an automatic restart the monitoring application and to trigger performance of various actions.

The medical systems and techniques described herein may implement geofencing technology for a computing service, which operates on a local and/or remote computing system and is configured to recognize certain events, such as when the patient is at home. Location data for the geographic area on which the patient's home resides may be programmed into a computing device at the home such that the patient entering the home (or simply being in the home) causes the computing device to identify the patient and trigger execution of logic for the monitoring application from the patient device(s) (or only the patient). Hence, a geofence can be configured for the home using geographic coordinates.

The medical systems and techniques described herein may dedicate a health computing service for such restarting/triggering. Restarting the monitoring application may further trigger, as one example action, a service request to the health monitoring service. Invoking an appropriate function call via the API may include initiating, as part of the function call, the service request from the patient account to a remote computing system of a health monitoring service. Restarting the monitoring application may be in response to a determination that a current patient location corresponds to the authenticated area, generating, as part of the function call, a new network connection with the health monitoring service or resuming, as part of the function call, a previous network connection with the health monitoring service.

A device of a local health monitoring service may be configured using a number of alternative (e.g., non-geofencing) technologies and as such, may be configured with a GPS receiver operative to receive coordinates of the patient at their current location. Based on a comparison between both sets of coordinates, a device of the local health monitoring service may determine when the patient is currently located in their home or residence clinic and then, responsive to the determination, restart the monitoring application and complete an authentication sequence with the health monitoring service (e.g., without supplying a credential). A different embodiment of the location data may be an indication (e.g., a message) that a current patient location and the patient's home's location are within a pre-defined proximity. One example of the indication may be communicated from a NFC module.

To illustrate by way of example, the medical system and techniques are configured to monitor the device interrogation and identify instances whether the device interrogation has been interrupted for any reason. In addition to datasets of patient data that are collected and stored by the medical device, the monitoring application may collect and store datasets of other patient data in the patient's mobile device, and then submit both datasets for the interrupted interrogation. By mitigating such interruptions and keeping the monitoring application operative and active, the medical system and techniques may achieve low latencies in an amount of elapsed time between a capture of a dataset and a transmission of that dataset for external (e.g., remote) storage.

As another example, the computing system running the health monitoring service may be instantly notified of device malfunctions or application disruption in addition to receiving secure notifications regarding health event detections (e.g., cardiac arrhythmias), confidential notifications, and other private data. These notifications are secured by virtue of the service having control over the geofenced area.

If the patient device is shut down and/or the monitoring application is disabled or closed, the computing device initiate a process (e.g., a wake-up process) to load into memory and execute logic of the monitoring application to run a main process and possibly, additional processes. The main process of the monitoring application performs a number of operations including at least one data submission of patient data from the patient device to the computing device of the clinic. The medical system may implement functionality on an API for prompting the main process to perform an operation, such as a transmission of the patient data for a scheduled data submission that is past due. The computing device at the clinic may invoke a function call to trigger (e.g., periodic) transmissions of patient data to facilitate a pending clinic appointment. If the local health monitoring service previously submitted a request for specific datasets of patient data and that request was not filled by the monitoring application, the computing device at the clinic may invoke a function call to trigger a transmission of a requested data submission that is past due otherwise late. Instead of disabled or closed, the monitoring application may be dormant, for example, where the main process is running on processing circuitry but restricted to a background according to another example. In yet another example, the main process may have raised a fault and is not respond to any inter-process communications.

The order and flow of the operation illustrated in FIG. 5 are examples. The steps of FIG. 5 can be performed in a different order or concurrently. For example, processing circuitry can determine that the patient is within an authenticated (e.g., geofenced) geographic area and that the monitoring application is inoperative concurrently. In other examples according to this disclosure, more or fewer thresholds may be considered. Further, in some examples, processing circuitry may perform or not perform the methods of FIG. 5, or any of the techniques described herein, as directed by a user, e.g., via external device 12 or computing devices 100. For example, a patient, clinician, or other user may turn on or off functionality for identifying changes in patient health (e.g., using Wi-Fi or cellular services) or locally (e.g., using an application provided on a patient's cellular phone or using a medical device programmer).

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

The following examples are a non-limiting list of clauses in accordance with one or more techniques of this disclosure.

Example 1. A method performed by processing circuitry of a computing device comprising: determining that a monitoring application, previously executed by the processing circuitry, is inoperative; receiving, from an input device of the computing device, input data indicative of a current location of a patient; determining whether the current location of the patient corresponds to an authenticated area of the patient; and in response to the determination that the current location corresponds to the authenticated area and to the determination that the monitoring application is inoperative, restarting the monitoring application.

Example 2. The method of Example 1, wherein receiving the input data comprises: capturing signal data over a wireless connection with a Global Positioning Service (GPS) receiver.

Example 3. The method of Example 1, wherein receiving the input data further comprises: capturing signal data over a network connection with a computing service.

Example 4. The method of Example 1, wherein receiving the input data further comprises: capturing signal data over a wireless connection with a beacon device.

Example 5. The method of Example 1, wherein receiving the input data further comprises: capturing signal data over a local connection with a medical device.

Example 6. The method of any of Examples 1-5, wherein determining that the current location corresponds to the authenticated area further comprises determining that the current location corresponds to a residence or a home of the patient.

Example 7. The method of any of Examples 1-6, wherein receiving the input data comprises receiving the input data in a message communicated by a device of a computing service.

Example 8. The method of any of Example 7, wherein the device is located within the authenticated area.

Example 9. The method of any of Example 7, wherein the device is located in an external location to the authenticated area.

Example 10. The method of any of Examples 1-6, wherein the computing service comprises a health monitoring service.

Example 11. The method of any of Examples 1-6, wherein the computing service comprises a location service.

Example 12. The method of any of Examples 1-11, wherein the computing service is operating on a computing system comprising at least one of a local computing system or a remote computing system.

Example 13. The method of any of Examples 1-12, wherein receiving the input data further comprises receiving, by the input device, a connection request to the device of the computing service in response to the connection request from that device, the method further comprising: communicating, by an output device of the computing service, an acknowledgement to the device of the computing service in response to the connection request from that device.

Example 14. The method of any of Example 1-13, wherein restarting the monitoring application further comprises: in response to the determination that the current patient location corresponds to the authenticated area, automatically completing an authentication sequence with a health monitoring service for accessing a patient account.

Example 15. The method of any of Examples 1-14, wherein restarting the monitoring application further comprises: in response to the determination that the current patient location corresponds to the authenticated area, initiating a function call to trigger execution of the monitoring application.

Example 16. The method of any of Examples 1-15, wherein restarting the monitoring application further comprises: initiating an active operating mode for the monitoring application as part of the function call, wherein the monitoring application is to run in a foreground of the computing device.

Example 17. The method of any of Examples 1-15, wherein restarting the monitoring application further comprises: submitting, as part of the function call, a credential for an authentication sequence of a patient account.

Example 18. The method of any of Examples 1-15, wherein restarting the monitoring application further comprises: overriding, as part of the function call, a requirement of a credential for an authentication sequence of a patient account.

Example 19. The method of any of Examples 1-15, wherein restarting the monitoring application further comprises: initiating, as part of the function call, a service request from the patient account to a remote computing system of a health monitoring service.

Example 20. The method of any of Examples 1-15, wherein restarting the monitoring application further comprises: in response to the determination that the current patient location corresponds to the authenticated area, generating, as part of the function call, a new network connection with a health monitoring service or resuming, as part of the function call, a previous network connection with the health monitoring service.

Example 21. The method of any of Examples 1-20, wherein restarting the monitoring application further comprises: in response to the input data, invoking a wake-up function to execute, via the processing circuitry, the monitoring application, wherein the monitoring application is operative to detect health events based on patient data.

Example 22. The method of any of Examples 1-21, wherein restarting the monitoring application further comprises: storing, in non-volatile memory, data generated by the monitoring application and currently stored in volatile memory.

Example 23. A computing device comprising: an input device; a memory; and processing circuitry configured to execute logic for a monitoring application stored in the memory, the processing circuitry further configured to: determine that a monitoring application, previously executed by the processing circuitry, is inoperative; receive, from an input device of the computing device, input data indicative of a current location of a patient; determine whether the current location of the patient corresponds to an authenticated area of the patient; and in response to the determination that the current location corresponds to the authenticated area and to the determination that the monitoring application is inoperative, restart the monitoring application.

Example 24. The computing device of Example 23, wherein the input device further comprises communication circuitry that includes a BLUETOOTH module communicatively coupled to a device of the health monitoring service for receiving data indicative of the current patient location.

Example 25. The computing device of any of Examples 23-24, wherein the input data is directed to a second application that is different from the monitoring application, wherein the second application is configured to invoke a function call causing the monitoring application to restart.

Example 26. The computing device of any of Examples 23-25 communicatively coupled to a device of the health monitoring service.

Example 27. The computing device of any of Examples 23-26, wherein the computing device is operative within an authentication area as an intermediate between a medical device for health event detection and a health monitoring service.

Example 28. A medical system comprising: processing circuitry executing logic stored in memory configured to: determine that a monitoring application, previously executed by the processing circuitry, is inoperative; receive, from an input device of the computing device, input data indicative of a current location of a patient; determine whether the current location of the patient corresponds to an authenticated area of the patient; and in response to the determination that the current location corresponds to the authenticated area and to the determination that the monitoring application is inoperative, restart the monitoring application.

Example 29. The medical system of Example 28, wherein a patient device is configured to be operative within an authentication area as an intermediate between a medical device for monitoring a patient and a health monitoring service, and the patient device comprises the input device, the output device, and the processing circuitry.

Example 30. The medical system of any of Examples 28-29, wherein the input data is directed to a second application that is different from the monitoring application, wherein the second application is configured to invoke a function call causing the monitoring application to restart.

Example 31. The medical system of any of Examples 28-30, wherein the medical device is communicatively coupled to the patient device, wherein the medical device comprises at least one of an implantable device, a wearable device, a pacemaker, a defibrillator, or a ventricular assist device (VAD) that comprises one or more sensors and sensing circuitry.

Example 32. A non-transitory computer-readable storage medium comprising program instructions that, when executed by processing circuitry of a medical device, cause the processing circuitry to: determine that a monitoring application, previously executed by the processing circuitry, is inoperative; receive, from an input device of the computing device, input data indicative of a current location of a patient; determine whether the current location of the patient corresponds to an authenticated area of the patient; and in response to the determination that the current location corresponds to the authenticated area and to the determination that the monitoring application is inoperative, restart the monitoring application.

Example 33. A method comprising any combination of the methods described herein.

Example 34. A system comprising processing circuitry configured to perform the method of any one or more of Examples 1-17.

Example 35. A non-transitory computer readable storage medium comprising program instructions configured to cause processing circuitry to perform the method of any one or more of Examples 1-17.

1. A computing device comprising:
an input device;
a memory; and
processing circuitry configured to execute logic for a monitoring application stored in the memory, the processing circuitry further configured to:
determine that a monitoring application, previously executed by the processing circuitry, is inoperative;
receive, from an input device of the computing device, input data indicative of a current location of a patient;
determine whether the current location of the patient corresponds to an authenticated area of the patient; and
in response to the determination that the current location corresponds to the authenticated area and to the determination that the monitoring application is inoperative, restart the monitoring application.

2. The computing device of claim 1, wherein the input device further comprises communication circuitry communicatively coupled to a device of a computing service for receiving data indicative of the current patient location, the computing service being, a health monitoring service or a location service.

3. The computing device of claim 1, wherein the input data is directed to a second application that is different from the monitoring application, wherein the second application is configured to invoke a function call causing the monitoring application to restart.

4. The computing device of claim 1, wherein the computing device is operative within an authentication area as an intermediate between a medical device for health event detection and a health monitoring service or location service.

5. The computing device of claim 1, wherein determining that the current location corresponds to the authenticated area further comprises determining that the current location corresponds to a residence or a home of the patient.

6. The computing device of claim 1, wherein receiving the input data comprises:
receiving the input data in a message communicated by a device of a computing service

7. The computing device of claim 1, wherein restarting the monitoring application further comprises one or more of:
in response to the determination that the current patient location corresponds to the authenticated area, automatically completing an authentication sequence with a health monitoring service for accessing a patient account,
in response to the determination that the current patient location corresponds to the authenticated area, initiating a function call to trigger execution of the monitoring application,
initiating an active operating mode for the monitoring application as part of the function call, wherein the monitoring application is to run in a foreground of the computing device,
submitting, as part of the function call, a credential for an authentication sequence of a patient account,
overriding, as part of the function call, a requirement of a credential for an authentication sequence of a patient account,
initiating, as part of the function call, a service request from the patient account to a remote computing system of a health monitoring service,
in response to the determination that the current patient location corresponds to the authenticated area, generating, as part of the function call, a new network connection with a health monitoring service or resuming, as part of the function call, a previous network connection with the health monitoring service,
in response to the input data, invoking a wake-up function to execute, via the processing circuitry, the monitoring application, wherein the monitoring application is operative to detect health events based on patient data, or
storing, in non-volatile memory, data generated by the monitoring application and currently stored in volatile memory.

8. A method performed by processing circuitry of a computing device comprising:
determining that a monitoring application, previously executed by the processing circuitry, is inoperative;
receiving, from an input device of the computing device, input data indicative of a current location of a patient;
determining whether the current location of the patient corresponds to an authenticated area of the patient; and
in response to the determination that the current location corresponds to the authenticated area and to the determination that the monitoring application is inoperative, restarting the monitoring application.

9. The method of claim 8, wherein receiving the input data comprises one or more of:
capturing signal data over a wireless connection with a Global Positioning Service (GPS) receiver;
capturing signal data over a network connection with a computing service;
capturing signal data over a wireless connection with a beacon device; or
capturing signal data over a local connection with a medical device.

10. The method of claim 8, wherein determining that the current location corresponds to the authenticated area further comprises determining that the current location corresponds to a residence or a home of the patient.

11. The method of claim 8, wherein receiving the input data comprises receiving the input data in a message communicated by a device of a computing service, the computing service being one of a health monitoring service or a location service.

12. The method of claim 11, wherein receiving the input data further comprises receiving, by the input device, a connection request to the device of the computing service in response to the connection request from that device, the method further comprising:
communicating, by an output device of the computing service, an acknowledgement to the device of the computing service in response to the connection request from that device.

13. The method of claim 8, wherein restarting the monitoring application further comprises one or more of:
in response to the determination that the current patient location corresponds to the authenticated area, automatically completing an authentication sequence with a health monitoring service for accessing a patient account,
in response to the determination that the current patient location corresponds to the authenticated area, initiating a function call to trigger execution of the monitoring application,
initiating an active operating mode for the monitoring application as part of the function call, wherein the monitoring application is to run in a foreground of the computing device,
submitting, as part of the function call, a credential for an authentication sequence of a patient account,
overriding, as part of the function call, a requirement of a credential for an authentication sequence of a patient account,
initiating, as part of the function call, a service request from the patient account to a remote computing system of a health monitoring service,
in response to the determination that the current patient location corresponds to the authenticated area, generating, as part of the function call, a new network connection with a health monitoring service or resuming, as part of the function call, a previous network connection with the health monitoring service,
in response to the input data, invoking a wake-up function to execute, via the processing circuitry, the monitoring application, wherein the monitoring application is operative to detect health events based on patient data,
storing, in non-volatile memory, data generated by the monitoring application and currently stored in volatile memory.

14. A non-transitory computer-readable storage medium comprising program instructions that, when executed by processing circuitry of a medical device, cause the processing circuitry to:
determine that a monitoring application, previously executed by the processing circuitry, is inoperative;
receive, from an input device of the computing device, input data indicative of a current location of a patient;
determine whether the current location of the patient corresponds to an authenticated area of the patient; and
in response to the determination that the current location corresponds to the authenticated area and to the determination that the monitoring application is inoperative, restart the monitoring application.

15. The computing device of claim 1, wherein restarting the monitoring application further comprises:
in response to the determination that the current patient location corresponds to the authenticated area, automatically completing an authentication sequence with a health monitoring service for accessing a patient account.

16. The computing device of claim 1, wherein restarting the monitoring application further comprises:
in response to the determination that the current patient location corresponds to the authenticated area, initiating a function call to trigger execution of the monitoring application.

17. The computing device of claim 1, wherein restarting the monitoring application further comprises:
initiating an active operating mode for the monitoring application as part of a function call, wherein the monitoring application is to run in a foreground of the computing device.

18. The computing device of claim 1, wherein restarting the monitoring application further comprises:
overriding, as part of a function call, a requirement of a credential for an authentication sequence of a patient account.

19. The computing device of claim 1, wherein restarting the monitoring application further comprises:
in response to the determination that the current patient location corresponds to the authenticated area, generating, as part of a function call, a new network connection with a health monitoring service or resuming, as part of the function call, a previous network connection with the health monitoring service.

20. The computing device of claim 1, wherein restarting the monitoring application further comprises:
in response to the input data, invoking a wake-up function to execute, via the processing circuitry, the monitoring application, wherein the monitoring application is operative to detect health events based on patient data.
